(19) United States
(12) Patent Application Publication
(10) Pub. No.: US 2009/0181419 A1
(43) Pub. Date: Jul. 16, 2009

(54) Method for Quantitatively Determining the Apolipoprotein AI Content in the HDL 2B Subfraction of HDL Cholesterol Subfractions

(76) Inventors: Frank Ruderman, San Rafael, CA (US); Christopher Boggess, San Francisco, CA (US); George Russell Warnick, Alameda, CA (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 12/199,780

(22) Filed: Aug. 27, 2008

Related U.S. Application Data
(60) Provisional application No. 60/966,267, filed on Aug. 27, 2007.

(51) Int. Cl. C12Q 1/02; G06F 7/00

(52) U.S. Cl. 435/29; 707/104.1; 707/E17.107; 707/E17.009

(57) ABSTRACT

The invention provides a method (e.g., a computer algorithm) for calculating a number of particles in a HDL subfraction. The method features the steps of: 1) measuring an initial distribution of HDL particles (e.g., a relative mass distribution) from a blood sample; 2) processing the initial distribution of HDL particles with a mathematical model to determine a modified distribution of HDL particles (e.g., a relative particle distribution); 3) determining a total apo-AI content value from a blood sample; and 4) analyzing both the modified distribution of particles and the total apo-AI content value to calculate the apo-AI content value in an HDL subfraction.

This application claims the benefit of priority to U.S. Provisional Patent Application Ser. No. 60/966,267, filed Aug. 27, 2007, which is herein incorporated by reference.

### FIELD OF THE INVENTION

The invention generally relates to the field of cardiovascular healthcare management. More particularly, the invention provides methods for identifying, measuring, and quantifying subfractions of high-density lipoprotein cholesterol (HDL) and protein content of such subfractions.

### BACKGROUND OF THE INVENTION

Although mortality rates for cardiovascular disease (CVD) have been declining in recent years, this condition remains the primary cause of death and disability in the United States for both men and women. In total, nearly 70 million Americans have a form of CVD, which includes high blood pressure (approximately 50 million Americans), coronary heart disease (12.5 million), myocardial infarction (7.3 million), angina pectoris (6.4 million), stroke (4.5 million), congenital cardiovascular defects (1 million), and congestive heart failure (4.7 million). Atherosclerotic cardiovascular disease (ASCVD), a form of CVD, can cause hardening and narrowing of the arteries, which in turn restricts blood flow and impedes delivery of vital oxygen and nutrients to the heart. Progressive atherosclerosis can lead to coronary artery, cerebral vascular, and peripheral vascular disease, which in combination result in approximately 75% of all deaths attributed to CVD.

Various lipoprotein abnormalities, including elevated concentrations of LDL and increased small, dense LDL subfractions, are causally related to the onset of ASCVD. Over time these compounds contribute to a harmful formation and build-up of atherosclerotic plaque in an artery's inner walls, thereby restricting blood flow. The likelihood that a patient will develop ASCVD generally increases with increased levels of LDL cholesterol, which is often referred to as “bad cholesterol.” Conversely, high-density lipoprotein cholesterol (referred to herein as “HDL”) can function as a “cholesterol scavenger” that binds cholesterol and transports it back to the liver for re-circulation or disposal. This process is called ‘reverse cholesterol transport’. A high level of HDL is therefore associated with a lower risk of heart disease and stroke, and thus HDL is typically referred to as “good cholesterol.”

A lipoprotein analysis (also called a lipoprotein profile or lipid panel) is a blood test that measures blood levels of LDL and HDL. One method for measuring HDL and LDL and some of their associated subfractions is described in U.S. Pat. No. 6,812,033, entitled “Method for identifying at-risk cardiovascular disease patients.” This patent, assigned to Berkeley HeartLab Inc. and incorporated herein by reference, describes a blood test based on gradient-gel electrophoresis (GGE). Gradient gels used in GGE are typically prepared with varying concentrations of acrylamide and can separate macromolecules according to mass with relatively high resolution compared to conventional electrophoretic gels. Using this technology, GGE can determine subfraction profiles of both HDL and LDL. For example, GGE can differentiate up to seven subfractions of LDL (referred to herein as LDL I, IIa, IIb, IIIa, IIIb, IVa, and IVb), and up to five subfractions of HDL (referred to herein as HDL 2b, 2a, 3a, 3b, 3c). Lipoprotein subfractions determined from GGE are also referred to as “sub-particles” or “particles” and correlate to results from a technique called analytic ultracentrifugation (AnUC), which is an established clinical research standard for lipoprotein subfractionation. (See generally, e.g., Cheung, M. C., et al., J Lipid Res (1991) 32:383-394; Berglund, L., et al., Am J Clin Nutr, (1999) 70:992-1000; Silverman, D. I., et al., Am J Med, (1993) 94:636-645; Schaefer, E. J., et al., Lipids, (1979) 14:511-522; DeLalla, O. F., et al., Am J Physiol, (1954) 179:333-337; Anderson, D. W., et al., Atherosclerosis, (1977) 29:161-179; Blanche, P. J., et al., Biochim Biophys Acta, (1981) 665:408-419; Verdery, R. B., et al., J Lipid Res, (1989) 30:1085-1095; Li, Z., et al., J Lipid Res, (1994) 35:1698-1711; and Cheung, M. C., et al., J Biol Chem, (1984) 259: 12201-12209).

Elevated levels of LDL IVb, a subfraction containing the smallest LDL particles, have been reported to have an independent association with arteriographic progression; a combined distribution of LDL IIIa and LDL IIIb typically reflects the severity of this trait.

Apolipoproteins are protein components of lipoproteins with three major functions: (1) maintaining the stability of lipoprotein particles; (2) acting as cofactors for enzymes that act on lipoproteins; and (3) removing lipoproteins from circulation by receptor-mediated mechanisms. The four groups of apolipoproteins are apolipoproteins A (Apo A), B (Apo B), C (Apo C) and E (Apo E). Each of the three groups A, B and C consists of two or more distinct proteins. These are for Apo A: Apo A-I, Apo A-II, and Apo A-IV, for Apo B: Apo B-100 and Apo B-48; and for Apo C: Apo-CI, Apo-CII and Apo-CIII. Apo E includes several isoforms.

Each class of lipoproteins includes a variety of apolipoproteins in differing proportions with the exception of LDL, which contains Apo B-100 as a sole apolipoprotein. Apo-AI and Apo-AII constitute approximately 90 percent of the protein moiety of HDL whereas Apo C and Apo E are present in various proportions in chylomicrons, VLDL, IDL and HDL. Apo B-100 is present in LDL, VLDL and IDL. Apo B-48 resides only in chylomicrons and so called chylomicron remnants (Kane, J. P., Method. Enzymol. 129:123-129 (1986)).

Apolipoproteins, such as Apo-B100 and Apo-AI are an essential part of lipid metabolism and are primary components of LDL and HDL lipoproteins, respectively. Apo-B100 and related compounds provide structural integrity to lipoproteins and protect hydrophobic lipids (i.e., non-water absorbing lipids) at their center. These proteins are recognized by receptors found on the surface of many of the body's cells and help bind lipoproteins to those cells to allow the transfer, or uptake, of cholesterol and triglyceride from the lipoprotein into the cells. Elevated levels of Apo-B100 correlate to elevated levels of LDL particles, and are also associated with an increased risk of coronary artery disease (CAD) and other cardiovascular diseases. Apo-AI is the major protein constituent of lipoproteins in the high density range (HDL subfractions). Apo-AI may also be the ligand that binds to a proposed hepatic receptor for HDL removal. A number of studies support the clinical sensitivity and specificity of Apo-AI as a negative risk factor for atherosclerosis (Avogaro, P. et al., Lancet, 1:901-903 (1979); Maciejko, J. J. et al., N. Engl. J. Med., 309:385-389 (1983)). Some investigators have also described Apo-AI/Apo-B100 ratio as a useful index of atherosclerotic risk (Kwiterovich, P. O. et al., Am. J. Cardiol., 69:1015-1021 (1992); Kuyl, J. M. and Mendelsohn, D., Clin. Biochem., 25:313-316 (1992)).

Each LDL cholesterol particle has an Apo-B100 molecule, and thus to a first approximation LDL particle number and Apo-B100 have a 1:1 correspondence. (See, US Published Patent Application No: 2007/0072303, incorporated by reference herein). In addition, elevated levels of Apo-B100, and other Apo-B proteins, are considered markers for determining an individual's risk of developing CAD when conjunctively compared to elevated small, dense LDL particles. While there may be some elevation of these values due to the inclusion of Apo-B100 from very low density lipoproteins (VLDL), this elevation is estimated to be less than 10% for triglyceride values of less than 200 mg/dL.

Similarly, the two heterologous subpopulations of HDL lipoprotein particles (LPA-I and LPA-I:A-II) contain at least one copy of Apo-AI. (Koren, E. et al. Clin. Chem., 33:38-43 (1987)). LPA-I particles contain Apo-AI but no Apo-AII, while LPA-I:A-II particles contain both apolipoproteins (Apo-AI and Apo-AII). HDL subpopulations (particles) are typically measured by established methods known in the art, such as analytical ultracentrifugation, GGE, enzyme immunoassay (Koren, E. et al. Clin. Chem., 33:38-43 (1987)) or electroimmunoassay (Atmeh, R. F. et al., Biochim. Biophys. Acta, 751:175-188 (1983)). As noted above, the importance of HDL has been emphasized by several studies which have demonstrated, for example, that LPA-I is a more active component in reverse cholesterol transport and, therefore, more anti-atherogenic than other lipoproteins (Puchois, P. et al., Atherosclerosis, 68:35-40 (1987); Fruchart, J. C. and Ailhaud, G., Clin. Chem., 38:793-797 (1992)). Thus, to a first approximation, HDL particle number and Apo-AI have a 1:1 correspondence, as Apo-AI is present in all HDL particles.

### SUMMARY OF THE INVENTION

In a first aspect, the invention provides a method (e.g., a computer algorithm) for calculating the Apo-AI content in a HDL 2b subfraction. The method features the steps of: (1) measuring an initial distribution of HDL particles (e.g. a relative mass distribution) from a blood sample; (2) processing the initial distribution of HDL particles with a mathematical model to determine a modified distribution (e.g., a relative particle distribution); (3) determining a total Apo-AI value from a blood sample; and (4) analyzing both the modified distribution of particles from (2), and the total Apo-AI value to calculate the Apo-AI content in a HDL 2b subfraction.

In one aspect, the invention provides a system for monitoring a patient that includes: (1) a database that stores blood test information describing, e.g., the Apo-AI content in a HDL 2b subfraction; (2) a monitoring device comprising systems that monitor the patient's vital sign information; (3) a database that receives vital sign information from the monitoring device; and (4) an Internet-based system configured to receive, store, and display the blood test and vital sign information.

These and other advantages of the invention will be apparent from the following detailed description and from the claims.

### BRIEF DESCRIPTION OF THE DRAWINGS

FIG. 1 depicts a flow chart describing an algorithm for calculating the apo-AI content in each HDL subfraction from a relative mass distribution of HDL subclasses.

FIG. 2 depicts a high-level schematic view of an internet-based system that collects and analyzes blood test information, such as apo-AI content within an HDL subfraction as determined using the algorithm presented in FIG. 1.

### DETAILED DESCRIPTION OF THE INVENTION

Reference is made to U.S. Provisional Patent Application Nos: 60/722,051; 60/721,825; 60/721,665; 60/721,756; and 60/721,617, each filed Sep. 29, 2005, and each incorporated by reference herein. Reference is also made to U.S. patent application Ser. No: 11/522,591, filed Sep. 18, 2006; U.S. patent application Ser. No: 11/522,650, filed Sep. 18, 2006; U.S. patent application Ser. No: 11/522,565, filed Sep. 18, 2006; U.S. patent application Ser. No: 11/522,562, filed Sep. 18, 2006; and U.S. patent application Ser. No. 11/522,589, filed Sep. 18, 2006, each of which is incorporated by reference herein. In addition, all references cited herein are incorporated by reference.

The invention provides advantages over known methods, particularly because it uses Apo-AI content to determine in the HDL 2b subfraction (the clinically relevant HDL measurement), rather than just a relative percentage of a mass distribution of HDL particles. For example, a patient's percent mass distribution of HDL 2b particles may remain unchanged, increase or decrease over time in response to aggressive lipid-lowering therapy, especially when the patient's total HDL cholesterol is significantly lowered by using a cholesterol-lowering compound. In contrast to a potential variable change in percent distribution of HDL subclasses, these therapies can raise Apo-AI content within a given subfraction, as determined by the method of this invention. A physician may use this information, in turn, to develop a specific cardiac risk reduction program for the patient targeting a quantifiable lipid-lowering therapeutic response. The determination of Apo-AI in the HDL 2b subfraction of a given patient can be highly useful in diagnosing heart disease and/or heart disease risk in subpopulation of patients.

The Apo-AI content in each HDL subfraction, taken alone or combined with other blood tests, may also be used in concert with an Internet-based disease-management system and a vital sign-monitoring device. This system can process information to help a patient comply with a personalized cardiovascular risk reduction program. For example, the system can provide personalized programs and their associated content to the patient through a messaging platform that sends information to a website, email address, wireless device, or monitoring device.

In one aspect, the invention provides a method (e.g., a computer algorithm) for calculating the Apo-AI content in a HDL 2b subfraction. The method features the steps of: (1) measuring an initial distribution of HDL particles (e.g. a relative mass distribution) from a blood sample; (2) processing the initial distribution of HDL particles with a mathematical model to determine a modified distribution (e.g., a relative particle distribution); (3) determining a total Apo-AI value from a blood sample; and (4) analyzing both the modified distribution of particles from (2), and the total Apo-AI value to calculate the Apo-AI content in a HDL 2b subfraction.

In certain embodiments, the mathematical model used in the algorithm analyzes at least one geometrical property of HDL particles (e.g., radius, diameter) within an HDL subfraction to determine a conversion factor. For example, the conversion factor can be derived from a ratio of surface areas for HDL particles within two subfractions. The conversion factor is determined before any processing, and is a constant for all patients. Once determined, the algorithm uses the conversion factor to convert the relative mass distribution into a relative particle distribution, which is then used to quantify the Apo-AI content in a HDL 2b subfraction.

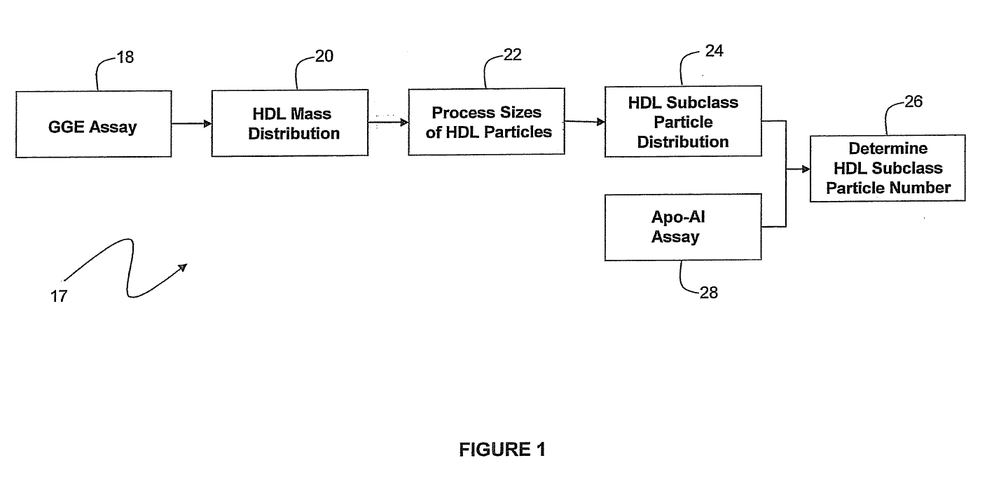

FIG. 1 depicts a non-limiting algorithm (17) that quantitatively determines the apo-AI content in each subfraction from the relative mass distribution (20). Analysis of a quantitative number of particles, as opposed to a relative mass distribution of particles, may additionally enable a medical professional design or alter an effective, customized cardiac risk reduction program for the patient, such as described in more detail below.

The algorithm (17) begins by processing inputs from an assay (e.g., a GGE assay (18)) to generate a relative mass distribution of HDL particles (20). An example of a GGE assay is described in U.S. Pat. No. 6,812,033, entitled ‘Method for identifying at risk cardiovascular disease patients’, the contents of which are incorporated herein by reference. The algorithm (17) processes the particle sizes corresponding to each subfraction (22) by assuming: i) all particles within the subfractions are spherical; and ii) the upper and lower diameters of particles in each subfraction are constant for all patients. This step of the algorithm (17) is described in more detail below. By processing the particle size, the algorithm (17) determines the relative surface area ratios for particles in each subfraction, and uses this value to convert the relative mass distribution into a relative particle distribution (24). The relative particle distribution describes the relative percentage of particles that correspond to each subfraction.

A separate branch of the algorithm (17) determines the total, quantitative number of HDL particles using an Apo-AI value measured with a separate assay (28). Once the Apo-AI value is determined, the algorithm (17) estimates the Apo-AI content in each subfraction (30). The algorithm then processes this value with the relative distribution of HDL particles (24) to quantitatively determine the apo-AI content in each sub-fraction (26).

In an embodiment, the technique(s) used to determine the amount of Apo A-I include any method known in the art such as, for example, immunological procedures using antibodies directed against Apo-AI, including radio-immunoassay (RIA), enzyme immunoassay (ELISA), competitive or capture systems, fluorescence immunoassay, radial immunodiffusion, nephelometry, turbidimetry and electroimmunoassay. (See, e.g., U.S. Pat. No. 5,814,467; U.S. Pat. No. 5,055,396; U.S. Pat. No. 7,098,036; U.S. Pat. No. 6,107,045; and WO 96/000903).

The algorithm described in FIG. 1 requires a calculation to determine the relative particle distribution from the relative mass distribution of HDL particles. To make this calculation, the algorithm assumes each HDL particle is spherical, and thus the particle's average surface area (SA) expressed by the equation:

SA=4π(r)²

Using values from any conventional analytic method for subfractionation of HDL particles (e.g., GGE assay, AnUC, etc.), a relative mass distribution of particles can be determined, which includes information for each subfraction, for example, upper particle diameter, lower particle diameter, median diameter, and mean radius. Using such information along with the above equation, the relative proportion of the surface areas of various HDL particles can be determined. The inverse of the surface area ratios yields a factor that converts the relative mass distribution of HDL particles to a corresponding relative particle distribution. Using this same methodology, the entire relative number distribution of HDL particles can be calculated from the relative mass distribution measured from a segmented GGE assay.

The algorithm measures the apo-AI content in each subfraction by multiplying percentages from the relative number distribution by the Apo-AI value as determined from a separate assay.

After determining this profile, in some embodiments, the algorithm can integrate with other software systems for disease management, such as those described in the US Provisional and Non-Provisional Applications referred to above and incorporated by reference.

In an aspect, the invention provides a system for monitoring a patient that includes: (1) a database that stores blood test information describing, e.g., the Apo-AI content in a HDL 2b subfraction; (2) a monitoring device comprising systems that monitor the patient's vital sign information; (3) a database that receives vital sign information from the monitoring device; and (4) an internet-based system configured to receive, store, and display the blood test and vital sign information. “Blood test information” as used herein, means information collected from one or more blood tests, such as a GGE-based test. In addition to a relative mass distribution of HDL particles, blood test information can include concentration, amounts, or any other information describing blood-borne compounds, including but not limited to total cholesterol, LDL (and subfraction distribution), HDL (and subfraction distribution), triglycerides, Apo B particle, Apo-AI, lipoprotein (a), Apo E genotype, fibrinogen, folate, HbA_{1c}, C-reactive protein, homocysteine, glucose, insulin, and other compounds. “Vital sign information” as used herein, means information collected from patient using a medical device, e.g., information that describes the patient's cardiovascular system. This information includes but is not limited to heart rate (measured at rest and during exercise), blood pressure (systolic, diastolic, and pulse pressure), blood pressure waveform, pulse oximetry, optical plethysmograph, electrical impedance plethysmograph, stroke volume, ECG and EKG, temperature, weight, percent body fat, and other properties.

As noted above, prior studies indicate that careful analysis of a patient's HDL subfractions alone, or in combination with analysis of LDL subfractions, can determine the relative risk for CAD. In certain embodiments the invention comprises an internet-based disease-management system that analyzes the number of HDL particles, and optionally LDL particles, measured in each subfraction, and in response designs a customized cardiac risk reduction program for the patient. The system can also provide personalized programs and their associated content to the patient through a messaging platform that sends information to a website, email address, wireless device, or monitoring device. Ultimately the disease-management system and messaging platform combine to form an interconnected, easy-to-use tool that can engage the patient, encourage follow-on medical appointments, and build patient compliance. These factors, in turn, can help the patient lower their risk for certain medical conditions, such as CVD.

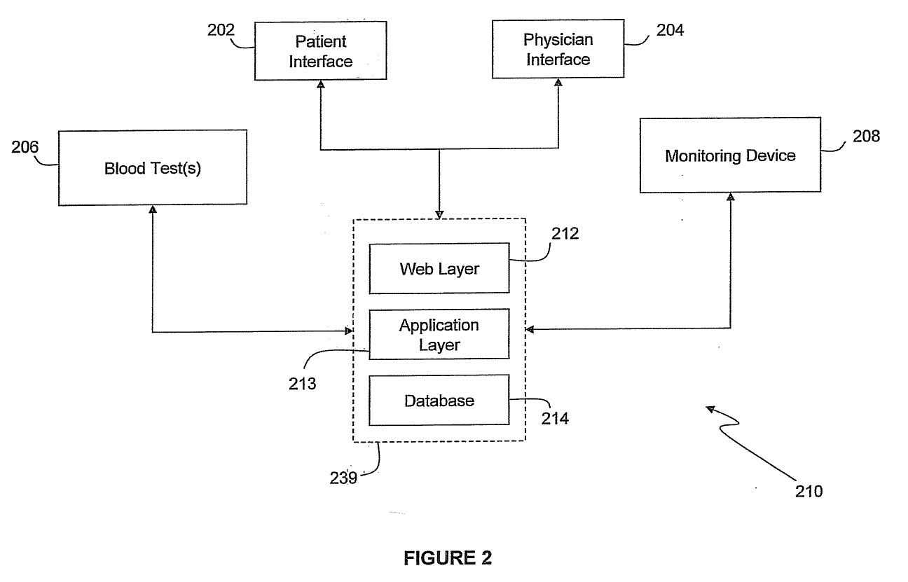

FIG. 2 depicts a non-limiting overview of an internet-based system (210) according to the invention that collects blood test information, such as information describing HDL subfractions (and optionally LDL subfractions), from one or more blood tests (206), and vital sign information (e.g., blood pressure, heart rate, pulse oximetry, and ECG information) from a monitoring device (208). Such a system is described, for example, in U.S. patent application Ser. No: 11/522,589, filed Sep. 18, 2006, incorporated herein by reference. The Internet-based system (210) features a web application (239) that manages software for a database layer (214), application layer (213), and interface layer (212) for, respectively, storing, processing, and displaying information. The web application (239) renders information from a single patient on a patient interface (202), and information from a group of patients on a physician interface (204). In certain embodiments, within the web application (239), the application layer (213) features information-processing algorithms that analyze the blood test and vital sign information stored in the database layer (214). Analysis of this information can yield a metabolic and cardiovascular risk profile that, in turn, can help the patient comply with a physician-directed cardiovascular risk reduction program. Specifically, based on this analysis, the interface layer (212) may render one or more web pages that describe a personalized program that includes reports and recommendations for diet, exercise, and lifestyle changes, along with content such as “heart-healthy” food recipes and news and reference articles. These web pages are available on both the patient (202) and physician (204) interfaces.

Other embodiments also fall within the scope of the invention. For example, the blood test and analysis method for determining the number of particles in each HDL cholesterol subfraction can be combined with other blood tests. In other embodiments, mathematical algorithms other than those described above can be used to analyze the HDL particles to convert a relative mass distribution into a relative particle distribution. In other embodiments, the total HDL value is measured directly, as opposed to being calculated from an Apo-AI value.

In still other embodiments, the web pages used to display information can take many different forms, as can the manner in which the data are displayed. Different web pages may be designed and accessed depending on the end-user. As described above, individual users have access to web pages that only chart their vital sign data (i.e., the patient interface), while organizations that support a large number of patients (e.g., doctor's offices and/or hospitals) have access to web pages that contain data from a group of patients (i.e., the physician interface). Other interfaces can also be used with the web site, such as interfaces used for: hospitals, insurance companies, members of a particular company, clinical trials for pharmaceutical companies, and e-commerce purposes. Vital sign information displayed on these web pages, for example, can be sorted and analyzed depending on the patient's medical history, age, sex, medical condition, and geographic location.

The web pages also support a wide range of algorithms that can be used to analyze data once it is extracted from the blood test information. For example, the above-mentioned text message or email can be sent out as an ‘alert’ in response to vital sign or blood test information indicating a medical condition that requires immediate attention. Alternatively, the message could be sent out when a data parameter (e.g. blood pressure, heart rate) exceeded a predetermined value. In some cases, multiple parameters can be analyzed simultaneously to generate an alert message. In general, an alert message can be sent out after analyzing one or more data parameters using any type of algorithm.

The system can also include a messaging platform that generates messages which include patient-specific content (e.g., treatment plans, diet recommendations, educational content) that helps drive the patient's compliance in a disease-management program (e.g. a cardiovascular risk reduction program), motivate the patient to meet predetermined goals and milestones, and encourage the patient to schedule follow-on medical appointments. Such a messaging system is described in co-pending U.S. patent application Ser. No: 11/522,562, filed Sep. 18, 2006, incorporated herein by reference.

In certain embodiments, the above-described methods, techniques, and systems can be used to characterize a wide range of diseases/disorders such as, for example, diabetes, heart disease, congestive heart failure, sleep disorders such as sleep apnea, asthma, heart attack and other cardiac conditions, stroke, Alzheimer's disease, and hypertension.

While the invention has been described above in terms of certain aspects and embodiments, the above description should not be viewed as limiting to the invention, as described by the claims

We claim:

1. A method for calculating the Apo-AI content in a HDL 2b subfraction comprising steps of:
(1) measuring an initial distribution of HDL particles from a blood sample;
(2) processing the initial distribution of HDL particles with a mathematical model to determine a relative particle distribution;
(3) determining a total Apo-AI value from a blood sample; and
(4) analyzing both the relative particle distribution and the total Apo-AI value to calculate the Apo-AI content in a HDL 2b subfraction.

2. The method of claim 1, wherein the initial distribution of HDL particles is a relative mass distribution.

3. The method of claim 2, wherein the processing step further comprises processing the relative mass distribution with a mathematical model that converts it to a relative particle distribution.

4. The method of claim 3, wherein the mathematical model analyzes at least one geometrical property of HDL particles within an HDL subfraction to determine a conversion factor.

5. The method of claim 4, wherein the geometrical property describes a size of the particle, and the conversion factor is derived from a ratio of a first surface area of a HDL particle within a first HDL subfraction, and second surface area of a HDL particle within a second HDL subfraction.

6. The method of claim 1, wherein the processing step further comprises processing the initial distribution of HDL particles with a mathematical model to determine a relative HDL particle distribution.

7. The method of claim 6, wherein the processing further comprises converting a relative mass distribution of HDL particles into a relative HDL particle distribution with the mathematical model.

8. The method of claim 1, wherein the determining step further comprises determining the total Apo-AI value or a derivative thereof.

9. The method of claim 8, further comprising the steps of: 1) measuring an Apo-AI value or a derivative thereof from a blood sample.

10. The method of claim 1, wherein the measuring step further comprises measuring an initial distribution of HDL particles from a blood sample using a segmented GGE-based assay.

11. The method of claim 1, wherein the measuring step further comprises measuring an initial distribution of HDL particles from analytical ultracentrifugation.

12. A method for calculating the apo-AI content in an HDL subfraction, comprising the steps of:
(1) measuring a relative mass distribution of HDL particles from a blood sample;
(2) processing the relative mass distribution of HDL particles with a mathematical model to determine a relative particle distribution of HDL particles;
(3) determining a total apo-AI content value from a blood sample; and
(4) analyzing both the relative particle distribution and apo-AI content value to calculate the apo-AI content in an HDL subfraction.

13. The method of claim 12, wherein the mathematical model analyzes at least one geometrical property of HDL particles within an HDL subfraction to determine a conversion factor.

14. The method of claim 13, wherein the geometrical property is a size of the particle, and the conversion factor is derived from a ratio of a first surface area of a HDL particle within a first HDL subfraction, and second surface area of a HDL particle within a second HDL subfraction.

15. The method of claim 12, wherein the determining step further comprises determining the total HDL particle number value from an Apo-AI value or a derivative thereof.

16. The method of claim 15, further comprising the step of: 1) measuring an Apo-AI value from a blood sample.

17. A system for monitoring a patient, comprising: a database that stores blood test information describing Apo-AI content in a HDL 2b subfraction; a monitoring device comprising systems that monitor the patient's vital sign information; a database that receives vital sign and exercise information from the monitoring device; and an Internet-based system configured to receive, store, and display the blood test, vital sign, and exercise information.
